(19) United States
(12) Patent Application Publication
(10) Pub. No.: US 2015/0343110 A1
(43) Pub. Date: Dec. 3, 2015

(54) PORTABLE AIR TREATMENT DEMONSTRATOR

(71) Applicant: Gregory Montgomery Williams, Memphis, TN (US)

(72) Inventor: Gregory Montgomery Williams, Memphis, TN (US)

(21) Appl. No.: 14/289,257

(22) Filed: May 28, 2014

(51) Int. Cl. A61L 9/20

(52) U.S. Cl. CPC A61L 9/20

(57) ABSTRACT

An air particle demonstration housing is used determine and compare air quality to ultraviolet (UV) treated air. The housing may utilize UV germicidal lamps in order to demonstrate the improvement that the UV lamp has on the air quality. First, the air quality within the building may be detected using a particle counter. Then air may be vacuumed into the box and treated with the UV lamps. The treated air from the box may be detected using a particle counter and compared with the untreated air quality.

### BACKGROUND OF THE INVENTION

The present invention relates to air quality and, more particularly, to a portable air treatment demonstrator.

Indoor air quality (IAQ) is a term which refers to the air quality within and around buildings and structures, especially as it relates to the health and comfort of building occupants. IAQ can be affected by gases, particulates, microbial contaminants (mold, bacteria), or any mass or energy stressor that can induce adverse health conditions. Source control, filtration, ultraviolet lamps and the use of to dilute contaminants are the primary methods for improving indoor air quality in most buildings and homes. Building dwellers and home owners may not detect reduced air quality because poor air quality cannot be seen, touched or smelled. Therefore, the user may be reluctant to acquire ultraviolet lamps when needed.

As can be seen, there is a need for a portable air treatment demonstrator having ultraviolet lamps.

### SUMMARY OF THE INVENTION

In one aspect of the present invention, a portable air treatment demonstrator comprises: a housing having an outer surface and an inner surface, wherein the inner surface forms an enclosure, wherein the housing comprises an opening formed through the housing leading into the enclosure; at least one ultraviolet lamp disposed within the enclosure of the housing; and a vacuum configured to push air into the enclosure passed the at least one ultraviolet lamp and out of the enclosure through the opening.

These and other features, aspects and advantages of the present invention will become better understood with reference to the following drawings, description and claims.

### BRIEF DESCRIPTION OF THE DRAWINGS

FIG. 1 is a front perspective view of the present invention;

FIG. 2 is a top plan view of the present invention; and

FIG. 3 is a perspective view of the present invention utilizing a particle counter.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes an air particle demonstration housing to determine and compare indoor air quality to ultraviolet (UV) treated air. The housing may utilize UV germicidal lamps in order to demonstrate the improvement that the UV lamp has on the air quality. First, the air quality within the building may be detected using a particle counter. Then air may be vacuumed into the box and treated with the UV lamps. The treated air from the box may be detected using a particle counter and compared with the untreated air quality.

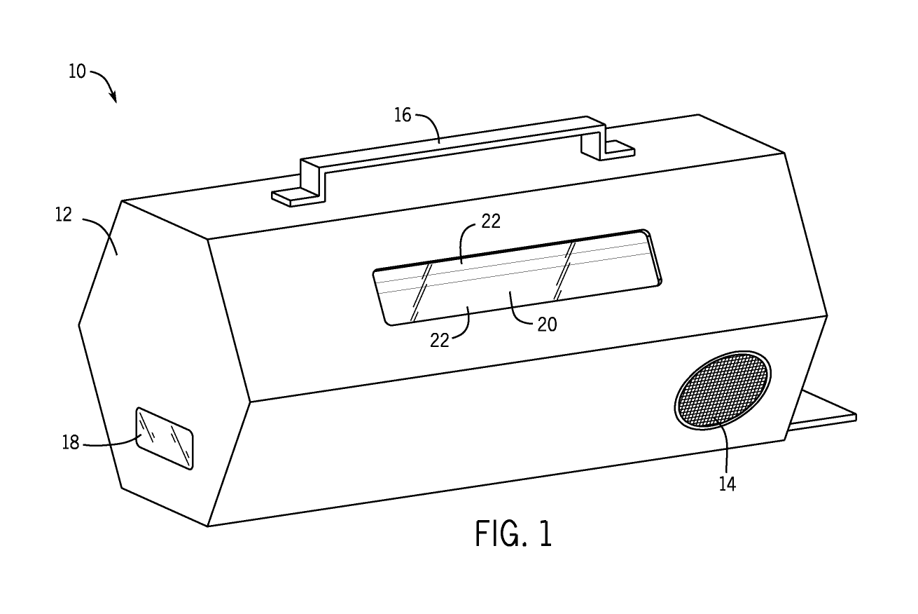

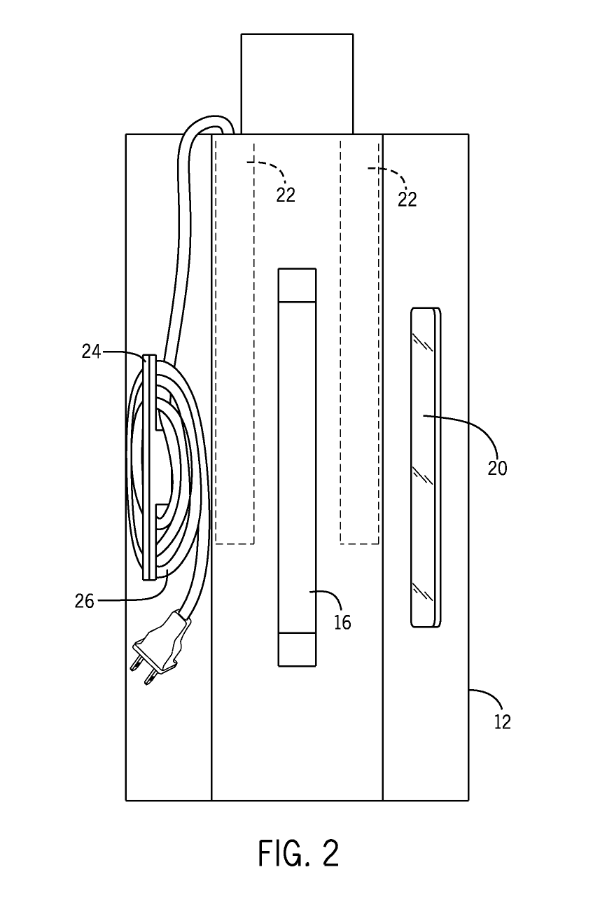

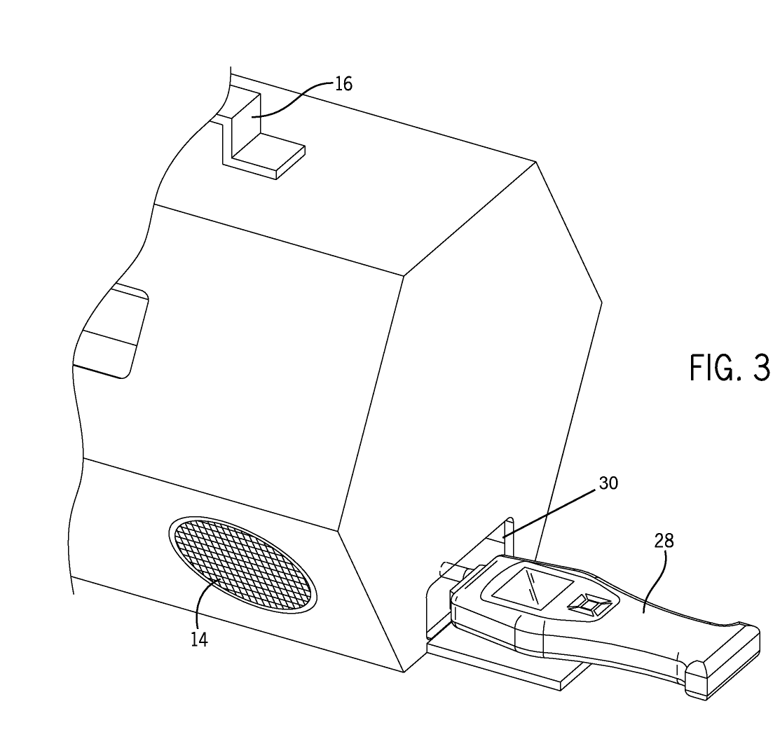

Referring to FIGS. 1 through 4, the present invention may include a portable air treatment demonstrator 10. The portable air treatment demonstrator 10 may include a housing 12. The housing 12 may include an outer surface and an inner surface. The inner surface forms an enclosure within the housing 12. The housing 12 may further include an opening 30 leading into the enclosure. In certain embodiments, a handle 16 may be attached to the housing 22 so that the housing 12 may be easily transported. The housing 12 may further include a window 20 built into it so that a user may easily look inside the housing 12.

The present invention may further include at least one ultraviolet lamp 22. The ultraviolet lamp 22 may be disposed within the enclosure of the housing 12. The ultraviolet lamp 22 may kill all contaminates within the housing 12. In certain embodiments, the present invention may include two ultraviolet lamps 22. The ultraviolet lamps 22 may be attached to the inner surface of the housing 12. In certain embodiments, the ultraviolet lamps 22 may be placed on the top portion of the inner surface of the housing 12.

The present invention may further include a vacuum 14. The vacuum may push air from outside of the housing 12 into the enclosure of the housing 12. The air may be directed within the housing 12 so that the air is exposed to the ultraviolet lamps 22. In certain embodiments, the vacuum 14 of the present invention may include a fan. The fan may be attached to the housing 12 and push air from outside of the housing 12 into the enclosure. Once the air has been treated, the air may be directed out of the opening 30.

The present invention may further include an air particle counter 28. The air particle counter 28 may count the amount of particles within the air. In certain embodiments, the air particle counter 28 may be built into the housing 12, and may thereby read the air particle count within the housing 12 and display the count on a monitor 18. Alternatively, the air particle counter 28 may be separate from the housing 12. Therefore, a user may test the air within the building using the separate air particle counter 28, and then place the air particle counter 28 in front of the opening 30 to test the treated air from the housing 12.

In certain embodiments, the present invention may further include a power cord 26 that is operatively connected to the ultraviolet lamps 22 and the vacuum 14, such as the fan. The power cord 26 may be plugged into an outlet to power the fan and the lamps 22. The power cord 26 may wrap around a cord holder 24 attached to the housing 12 when not in use. In certain embodiments, there may be a power button. The power button may control both the power to the ultraviolet lamps 22 and the fan. Alternatively, a first power button may control the ultraviolet lamps 22 and a second power button may control the fan. Therefore, if the present invention includes a built in air particle counter 30, the fan may be turned on first to demonstrate the air quality within the building, and then the ultraviolet lamp 22 may be turned on second to display the air treated by the ultraviolet lamp 22.

A method of demonstrating a difference in air quality may include the following. When using a separate particle counter, the user may first detect the particle count within the building. The user may then power the vacuum and the ultraviolet lamps. The vacuum may push the air into the housing and the ultraviolet lamps may treat the air. The treated air may exit through the opening, and the user may detect the particle count of the treated air. The difference of the air and the treated air may be shown to a potential client. Alternatively, if the air particle counter is built in, the user may power the vacuum and read the particle count displayed from the monitor 18. The user may then power the ultraviolet lamps. The treated air particle count may then be displayed from the monitor 18.

It should be understood, of course, that the foregoing relates to exemplary embodiments of the invention and that modifications may be made without departing from the spirit and scope of the invention as set forth in the following claims.

What is claimed is:

1. A portable air treatment demonstrator comprising:
a housing having an outer surface and an inner surface, wherein the inner surface forms an enclosure, wherein the housing comprises an opening formed through the housing leading into the enclosure;
at least one ultraviolet lamp disposed within the enclosure of the housing; and
a vacuum configured to push air into the enclosure passed the at least one ultraviolet lamp and out of the enclosure through the opening.

2. The portable air treatment demonstrator of claim 1, wherein the vacuum comprises a fan attached to the housing.

3. The portable air treatment demonstrator of claim 1, wherein the at least one ultraviolet lamp comprises two ultraviolet lamps attached to the inner surface of the housing.

4. The portable air treatment demonstrator of claim 2, further comprising a power cord operatively connected to the ultraviolet lamp and the fan.

5. The portable air treatment demonstrator of claim 1, further comprising a handle attached to the housing.

6. The portable air treatment demonstrator of claim 1, further comprising a window mounted to the housing.

7. The portable air treatment demonstrator of claim 1, further comprising an air particle counter.

8. The portable air treatment demonstrator of claim 7, wherein the particle counter is built into the housing, and is configured to read the air particle count within the housing and display the count on a monitor.

9. The portable air treatment demonstrator of claim 8, further comprising a first power button for the vacuum and a second power button for the ultraviolet lamp.

10. The portable air treatment demonstrator of claim 7, wherein the air particle counter is separate from the housing.
